(19) United States
(12) Patent Application Publication
(10) Pub. No.: US 2011/0201935 A1
(43) Pub. Date: Aug. 18, 2011

(54) 3-D ULTRASOUND IMAGING

(75) Inventors: Antoine Collet-Billon, Paris (FR); James Jago, Seattle, WA (US); Alasdair Dow, Snohomish, WA (US); Lisa Pumphrey, Kenmore, WA (US)

(73) Assignee: KONINKLIJKE PHILIPS ELECTRONICS N.V., Eindhoven (NL)

(21) Appl. No.: 13/124,919

(22) PCT Filed: Oct. 15, 2009
(86) PCT No.: PCT/IB09/54548

§ 371 Date: Apr. 19, 2011

Related U.S. Application Data
(60) Provisional application No. 61/107,392, filed on Oct. 22, 2008.

(51) Int. Cl. A61B 8/14

(52) U.S. Cl. 600/443

(57) ABSTRACT

In an ultrasound imaging system (UIS), an ultrasound scanning assembly (USC) provides volume data (VD) resulting from a three-dimensional scan of a body (BDY). A feature extractor (FEX) searches for a best match between the volume data (VD) and a geometrical model (GM) of an anatomical entity. The geometrical model (GM) comprises respective segments representing respective anatomic features. Accordingly, the feature extractor (FEX) provides an anatomy-related description (ARD) of the volume data (VD), which identifies respective geometrical locations of respective anatomic features in the volume data (VD). In a preferred embodiment, a slice generator (SLG) generates slices (SX) from the volume data (VD) based on the anatomy-related description (ARD) of the volume data (VD).

### FIELD OF THE INVENTION

An aspect of the invention relates to an ultrasound imaging system that is capable of carrying out a three-dimensional (3-D) ultrasound scan, which produces volume data. The ultrasound imaging system may be helpful in, for example, fetal examinations, in particular detection of structural anomalies in a fetal heart. Other aspects of the invention relate to a method of ultrasound imaging, and a computer program product.

### BACKGROUND OF THE INVENTION

A 3-D ultrasound scan typically involves emitting ultrasound waves that illuminate, as it were, a particular volume within a body, which may be designated as target volume. This can be achieved, for example, by emitting ultrasound waves at multiple different angles. Volume data is obtained by receiving and processing reflected waves. The volume data is a representation of the target volume within the body. The volume data can be displayed on a display device in a fashion that provides a three-dimensional representation, which gives an impression of width, height, and depth. In obstetric applications, it is possible to obtain a photo- or film-like image of a fetus with surface details that delineate facial, limbs and body features. This allows prospective parents to see and appreciate what physicians see.

3-D ultrasound scanning is particularly useful in obstetric applications from a diagnostic point of view. For example, the volume data can entirely capture a fetal heart. Arbitrary slices can be taken from the volume data and visualized on the display device. Slicing can thus provide two-dimensional (2-D) views at any orientation in the fetal heart, which may be difficult to achieve manually, or cannot be achieved at all. Moreover, 3-D ultrasound scanning allows views that are less operator-dependent compared with 2-D ultrasound scanning, where obtaining so-called standard views requires a high degree of skill. The volume data may be stored so that the physician may obtain standard views after a 3-D ultrasound scan of a patient and the patient is discharged. In fetal heart examination, standard views allow detection of structural anomalies, which are mostly congenital heart defects (CHD). Typical standard views are the so-called four-chamber view and views to assess left ventricle and right ventricle outflow tracts. However, finding such standard views that allow a reliable diagnosis still requires a relatively high level of skill. Moreover, this process is relatively error-prone and time-consuming.

United States patent application published under number US 2005/0004465-A1 describes a method for use in a medical-imaging environment. In the method, a reference plane is obtained for a particular body organ, which can be used as a baseline from which to obtain other planes of interest. The reference plane can be a standard representative plane that is relatively easy to obtain on 2-D ultrasonography, such as the four-chamber view plane of the fetal heart. A 3-D ultrasound imaging apparatus can then be used to acquire a volume of tissue starting, for example, from the level of, or with respect to, the reference plane. Spatial mathematical relationships of standardized planes in relation to the reference plane are provided for various fetal, neonatal and adult organs. Software and/or hardware utilized by a general purpose computer and/or standard sonography equipment may then utilize one or more of the mathematical relationships to display one or more of the standardized planes.

### SUMMARY OF THE INVENTION

There is a need for an improved ultrasound imaging system, which allows a quicker and more reliable analysis of volume data.

In order to better address this need, the following points have been taken into consideration. The method described in the aforementioned patent application suffers from several limitations. The method relies on an operator's ability to obtain a reference view that is sufficiently good, in particular in terms of its geometrical location. In case the reference view is imprecise, views that are obtained on the basis of the spatial mathematical relationships will also be imprecise and thus deviate from standard views that allow reliable diagnosis. Moreover, obtaining a reference view that is sufficiently good can be time consuming, in particular for a less experienced operator. In addition, the method requires manual manipulations, such as, for example, user clicks, translation or rotation in order to obtain the required standard views. These manipulations are also subject to possible errors and can be time consuming too.

The method described in the aforementioned patent application involves spatial mathematical relationships that are based on a priori knowledge and apply to a mean normal organ, such as, for example, a mean normal fetus heart. However, there may be significant variability in terms of geometric properties of the organ concerned, even in normal cases. In case of fetal examination, there may still be significant variability even if an adjustment for gestational age is made. It is possible to deal with this variability by presenting multiple candidate views from which the operator may select a standard view. However, this may be confusing for the operator, and may therefore lead to errors. Moreover, such an approach is rather inefficient.

In accordance with an aspect of the invention, an ultrasound imaging system comprises an ultrasound scanning assembly that provides volume data resulting from a three-dimensional scan of a body. More specifically, the ultrasound imaging system further comprises a feature extractor that searches for a best match between the volume data and a geometrical model of an anatomical entity. The geometrical model comprises respective segments representing respective anatomic features. Accordingly, the feature extractor provides an anatomy-related description of the volume data, which identifies respective geometrical locations of respective anatomic features in the volume data.

The anatomy-related description can be used to advantage in processing steps that are applied to the volume data for the purpose of analyzing this data and establishing a diagnosis. The anatomy-related description may guide, as it were, such processing steps. Identifying anatomic features is no longer a process that substantially depends on operator abilities and that requires relatively sophisticated skills. Standard views that are obtained from the volume data will be less operator dependent, and can be obtained with less skill required on the part of the operator, which allows a more reliable diagnosis. For example, in obstetric applications, this will typically result in a higher probability of detecting structural anomalies in a fetal heart. There is less risk that such anomalies remain undetected. Moreover, the anatomy-related description allows speeding up a process of manipulating and analyzing the volume data.

An implementation of the invention advantageously comprises one or more of the following additional features, which are described in separate paragraphs that correspond with individual dependent claims.

The ultrasound imaging system preferably comprises a slice generator for generating slices from the volume data based on the anatomy-related description of the volume data.

The feature extractor preferably provides a match-failure indication in case a smallest matching error that can be found between the volume data and the geometrical model exceeds a given threshold.

The ultrasound imaging system preferably comprises a user interface that allows an operator to define a minimization criterion that is to be used in finding the best match between the volume data and the geometrical model.

The feature extractor preferably obtains a reference slice that serves as a starting point in finding the best match between the volume data and the geometrical model.

The ultrasound imaging system preferably comprises a processor that generates segment-related graphical information from the anatomy-related description, and that overlays the segment-related graphical information on a slice that has been generated from the volume data.

A processor, which may be the aforementioned processor, preferably determines at least one axis of rotation for a slice on the basis of the anatomy-related description. Such an axis of rotation can be displayed in association with the slice.

A processor, which may be the aforementioned processor, preferably generates annotations for a slice on the basis of the anatomy-related description. These annotations can be stored in association with the slice.

The feature extractor preferably applies a deformation to the geometrical model in finding the best match between the volume data and the geometrical model.

The feature extractor preferably obtains a maximum degree of deformation that can be applied to the geometrical model in finding the best match between the volume data and the geometrical model.

The ultrasound scanning assembly preferably provided an indication of a time position within a biological cycle to which the volume data pertains. The feature extractor can then apply the geometrical model as a function of the indication of the time position.

A detailed description, with reference to drawings, illustrates the invention summarized hereinbefore as well as the additional features.

### BRIEF DESCRIPTION OF THE DRAWINGS

FIG. 1 is a block diagram that illustrates an ultrasound imaging system.

FIGS. 2A and 2B are flow chart diagrams that illustrate a series of steps that the ultrasound imaging system can carry out.

### DETAILED DESCRIPTION OF THE INVENTION

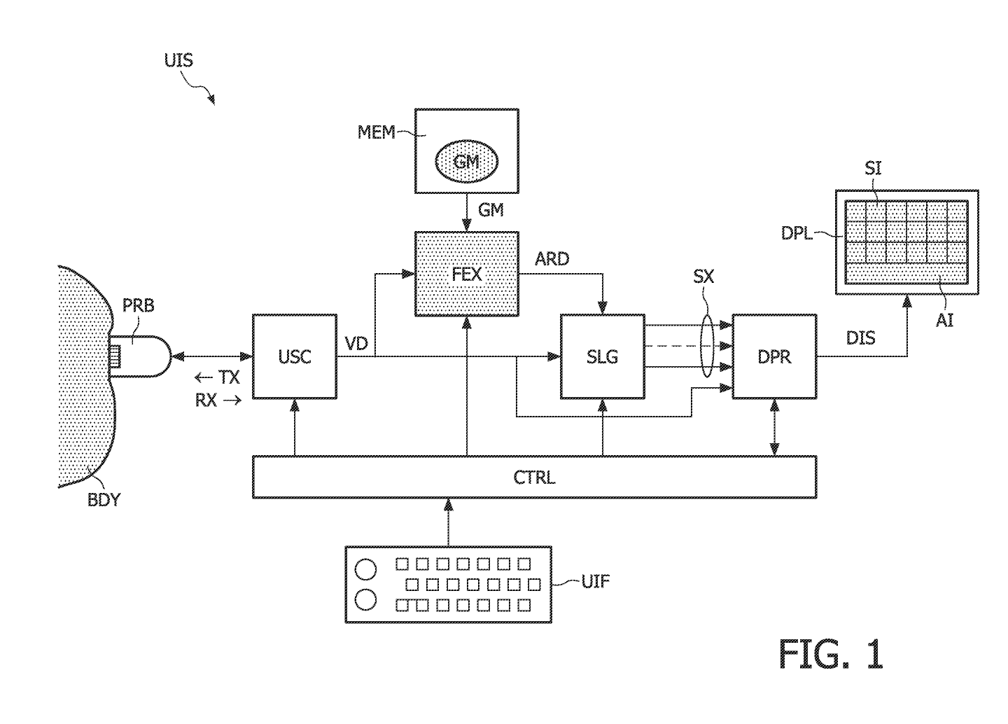

FIG. 1 illustrates an ultrasound imaging system UIS, which capable of carrying out a 3-D ultrasound scan. The ultrasound imaging system UIS comprises various functional entities that constitute an ultrasound imaging acquisition-and-processing path: a probe PRB, an ultrasound scanning assembly USC, a feature extractor FEX, a slice generator SLG, and a display processor DPR. The probe PRB may comprise, for example, a two-dimensional array of piezoelectric transducers. The ultrasound scanning assembly USC may comprise an ultrasound transmitter and an ultrasound receiver, which may each include a beam-forming module. The ultrasound scanning assembly USC may further comprise one or more filter modules, a so-called B-mode processing module, and a Doppler-mode processing module.

The feature extractor FEX may be implemented by means of, for example, a set of instructions that has been loaded into a programmable processor. In such a software-based implementation, the set of instructions defines operations that the feature extractor FEX carries out, which will be described hereinafter. The same holds for other functional entities, such as, for example, the slice generator SLG, the display processor DPR, as well as one or more modules that functionally belong to the ultrasound scanning assembly USC. Each of these may also be implemented by means of a set of instructions, a software module, which has been loaded into a programmable processor.

The ultrasound imaging system UIS further comprises a memory MEM in which a geometrical model GM of a fetal heart is stored. The memory MEM may be in the form of, for example, a hard disk or a solid-state memory. The memory MEM may comprise other data, such as, for example, the aforementioned set of instructions that implement the feature extractor FEX, as well as other program modules. The feature extractor FEX may use the geometrical model GM, as will be described hereinafter.

The ultrasound imaging system UIS further comprises a display device DPL, a controller CTRL, and a user interface UIF. The controller CTRL may be in the form of, for example, a suitably programmed processor. The user interface UIF may comprise physical elements, such as, for example, various alphanumerical keys, knobs, and a mouse or trackball. However, the user interface UIF may also comprise software components, which the controller CTRL carries out. For example, a software component may cause the display device DPL to display a menu from which an operator may select an item by pressing a particular key or by moving a cursor to the item as displayed.

The ultrasound imaging system UIS basically operates as follows. It is assumed that the probe PRB is in contact with a body BDY of a pregnant woman, who is carrying a fetus. The ultrasound scanning assembly USC applies a set of transmission signals TX to the probe PRB. This causes the probe PRB to emit ultrasound waves into the body BDY that illuminate, as it were, a target volume comprising a fetal heart. To that end, the probe PRB may emit, for example, ultrasound waves at multiple different angles. Alternatively, the set of transmission signals TX may cause the probe PRB to emit a relatively broad beam, which may be designated as a “fat” beam.

The probe PRB receives reflections of the ultrasound waves, which occur in the target volume within the body BDY. In response to these received reflections, the probe PRB provides a set of reception signals RX. The ultrasound scanning assembly USC processes the set of reception signals RX so as to obtain volume data VD. The volume data VD may be in the form of, for example, so-called B-mode 3-D image, or a 3-D Doppler-based image, which may comprise color information representing speed of movement. The volume data VD is typically composed of so-called voxels, which are elementary units similar to pixels, which constitute elementary units of a 2-D image.

The feature extractor FEX provides an anatomy-related description ARD of the volume data VD. To that end, the feature extractor FEX uses the geometrical model GM of the fetal heart that is stored in the memory MEM. In a manner of speaking, the feature extractor FEX tries to find a best match between the geometrical model GM and the volume data VD. This will be described in greater detail hereinafter. Once the best match has been found, the feature extractor FEX can define various segments in the volume data VD on the basis of the geometrical model GM. These various segments correspond with respective anatomic features of the fetal heart, and searches, for example, the heart chambers, the main vessels (thoracic aorta, thoracic arch, pulmonary arteries, vena cavae . . . ), the septa, the large valves. The anatomy-related description ARD identifies these segments in the volume data VD.

The slice generator SLG generates slices SX from the volume data VD. The anatomy-related description ARD determines where and how slices SX are generated from the volume data VD. The anatomy-related description ARD guides, as it were, the slice generator SLG in generating the slices SX so that these substantially correspond with a set of standard views of the fetal heart. These standard views, which are two-dimensional, may comprise a so-called 4-chamber view, and views to assess left ventricle and right ventricle outflow tracts. These standard views facilitate a diagnostic analysis of the volume data VD and improve reliability thereof. The aforementioned standard views may reveal congenital heart defects. Other views beyond the standard views, such as the ductal arch view, 5-chamber view, 3-vessel view, etc, may also be generated by the slice generator SLG.

The display processor DPR generates display images DIS that typically comprise a visual representation of the slices SX that the slice generator SLG has generated from the volume data VD. Each slice may be visualized, for example, by means of an individual sub-image SI in a display image DIS. Respective sub-images representing respective slices SX may be displayed side-by-side in the form of a matrix, as illustrated in FIG. 1, or any other form that the operator desires. The display image DIS may also comprise a full screen visual representation of a slice. A visual representation of a slice may further comprise various annotations that indicate anatomic features of the fetal heart. Such annotations may be generated automatically, as will be described in greater detail hereinafter.

The display image DIS may further comprise additional information AI relating to, for example, the location, the orientation, and the spacing of the slices SX. The display image DIS may further comprise a visual representation of the volume data VD, which may constitute an additional sub-image. As indicated hereinbefore, this visual representation may comprise additional elements that indicate actual or desired locations and orientations of slices SX within the volume data VD that are currently visualized, or that needs to be visualized, respectively.

The ultrasound imaging system UIS may acquire volume data VD at several successive instants in time. This provides a temporal dimension, which allows a slice SX to be displayed as a video on the display device DPL rather than as a still picture. Accordingly, a cardiac cycle of a fetal heart can be captured and visualized. Volume data VD, which is acquired at a particular instant, represents the fetal heart at a particular temporal position in the cardiac cycle, such as, for example, diastole or systole. The ultrasound scanning assembly USC, or another functional entity of the ultrasound imaging system UIS, is preferably arranged to provide an indication of the temporal position in the cardiac cycle for volume data VD.

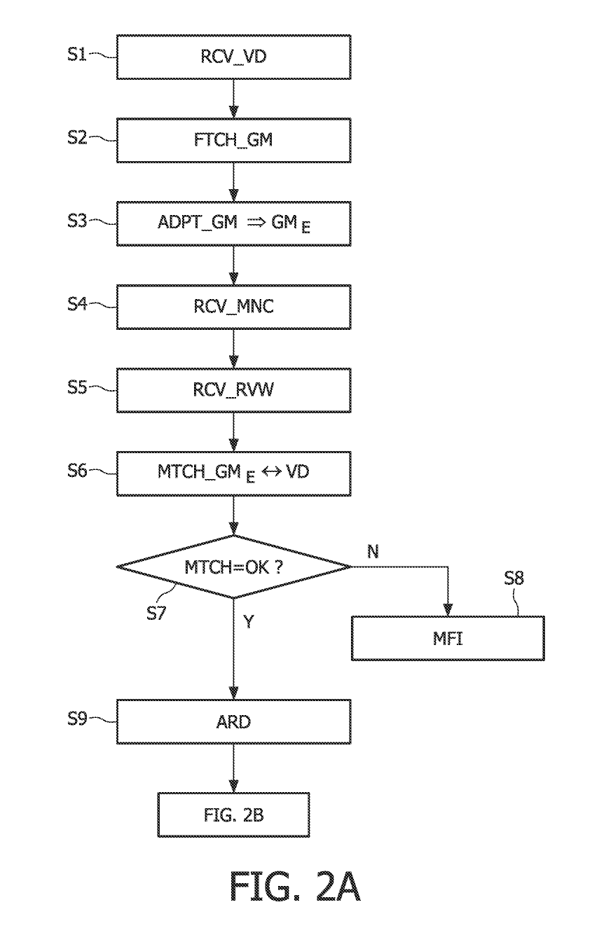

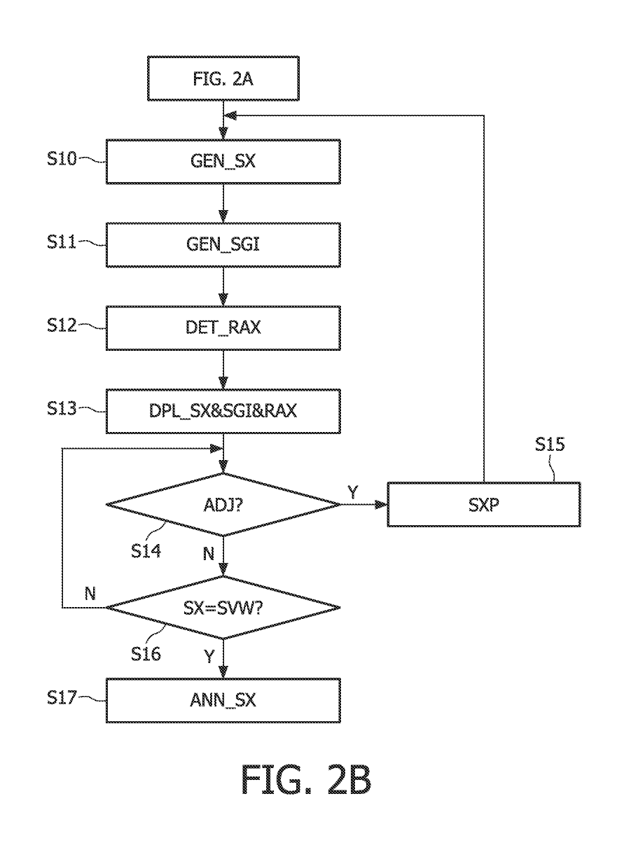

FIGS. 2A and 2B illustrate a series of steps S1-S15 that the ultrasound imaging system UIS may carry out in order to provide an anatomy-related description ARD. As mentioned hereinbefore, the feature extractor FEX, as well as other functional entities, may be implemented by means of a programmable processor. FIGS. 2A and 2B may therefore be regarded as a flowchart representation of a software program, that is, a set of instructions, which enables the programmable processor to carry out various operations described hereinafter with reference to FIG. 2.

In step S1 (RCV_VD), the feature extractor FEX receives the volume data VD that the ultrasound scanning assembly USC provides following a 3-D ultrasound scan. As mentioned hereinbefore, the volume data VD may be in the form of a 3-D image composed of voxels, which are elementary units similar to pixels, which constitute elementary units of a 2-D image. The volume data VD may comprise B-mode information, or Doppler information relating to speed of movement, or a combination of these types of information, as well as other information obtained by means of the 3-D ultrasound scan.

In step S2 (FTCH_GM), the feature extractor FEX fetches the geometrical model GM of the fetal heart that is stored in the memory MEM as illustrated in FIG. 1. The geometrical model GM may be regarded as a three-dimensional structure that comprises various segments. A segment represents a particular anatomic feature that has a specific shape and size, as well as a specific geometrical location with respect to other segments. The geometrical model GM may be in the form of a set of data elements, each relating to a particular anatomic feature and defining geometrical properties of that feature. To that end, a data element may comprise various fields, such as, for example, a field that identifies the anatomic feature to which the data element relates, and a series of fields that define the geometrical outline of the anatomic feature by means of, for example, a set of coordinates of a three-dimensional Cartesian coordinate system.

The geometrical model GM preferably comprises a temporal dimension. That is, the geometrical model GM preferably defines a series of three-dimensional structures that are segment-based, each of which pertains to a particular temporal position in the cardiac cycle, such as, for example, diastole or systole. A data element that relates to a particular anatomic feature may thus define geometrical properties of that feature for several different temporal positions in the cardiac cycle. To that end, the data element may comprise one or more fields defining variations in the geometrical properties of the anatomic feature concerned as a function of temporal position in the cardiac cycle.

In step S3 (ADPT_GM [symbol] GM_{E}), the feature extractor FEX may adapt the geometrical model GM so as to obtain an effective geometrical model GM_{E} that will be used for generating the anatomy-related description ARD. To that end, the feature extractor FEX, or the controller CTRL, may cause the display device DPL to display a message inviting an operator to specify one or more parameters that concern the fetus such as, for example, gestational age. The operator may specify such a parameter by means of the user interface UIF. The feature extractor FEX may then compare the one or more parameters that the operator has specified with one or more standard parameters that apply to the geometrical model GM. The feature extractor FEX may then adapt the geometrical model GM if needed. For example, the gestational age of the fetus may significantly differ from the gestational age that applies to the geometrical model GM. The feature extractor FEX will then adapt the geometrical model GM accordingly.

In case the geometrical model GM comprises a temporal dimension, the feature extractor FEX uses the indication of the temporal position in the cardiac cycle mentioned hereinbefore to obtain the effective geometrical model GM_{E}. Accordingly, the effective geometrical model GM_{E} will be adapted so that it best represents the fetal heart at the temporal position in the cardiac cycle concerned. This contributes to a process of matching the effective geometrical model GM_{E} with the volume data VD of interest. There is a greater probability that this matching process, which will be described in greater detail hereinafter, will be successful. Moreover, more precise results from this matching process can be obtained, which finally contribute to a reliable diagnosis as will be apparent from the description hereinafter.

In step S4 (RCV_MNC), the feature extractor FEX obtains a minimization criterion that is to be applied in generating the anatomy-related description ARD. The minimization criterion defines a process of finding a best match between the effective geometrical model GM_{E} and the volume data VD. Minimization criterion may, for example, consist of maximization of mutual information. The minimization criterion may be predefined. In that case, the feature extractor FEX may fetch the minimization criterion from a memory MEM, or the minimization criterion may be built in, as it were, in the set of instructions that implement the feature extractor FEX. Alternatively, the minimization criterion may be operator-defined. In that case, the feature extractor FEX, or the controller CTRL, may cause the display device DPL to display a message inviting an operator to specify the minimization criterion. The message may be in the form of, for example, a menu of various different minimization criteria from which the operator may select one by means of the user interface UIF.

In step S5 (RCV_RVW), the feature extractor FEX receives a geometrical definition of a reference slice within the volume data VD, which may correspond with a standard view. The reference slice, or rather, the geometrical definition thereof, may be used as a starting point in the process of finding a best match between the effective geometrical model GM_{E} and the volume data VD. Defining such a starting point may speed up the process or make it more reliable, or both. The reference slice may be, for example, the aforementioned 4-chamber view of the fetal heart. The operator may scan through the volume data VD so as to obtain an appropriate 4-chamber view that may serve as a reference. In that case, the operator provides the definition of the reference slice. This, however, requires a relatively high level of skill. In a more preferred approach, the feature extractor FEX may automatically search for and identify a slice that provides the standard view of interest.

In step S6 (MTCH_GM_{E} [symbol] VD), the feature extractor FEX tries to match the effective geometrical model GM_{E} with the volume data VD in accordance with the applicable minimization criterion. To that end, the feature extractor FEX may apply various geometrical manipulations to the geometrical model GM or the volume data VD, or both. These geometrical manipulations may include, for example, translation, rotation, and scaling. The feature extractor FEX may further deform the effective geometrical model GM_{E} in a smooth fashion so as to align a smoothly deformed geometrical model thus obtained with the volume data VD. There will typically be a maximum degree of deformation that may be applied to that effect. The maximum degree of deformation may be predefined or specified by the operator. In the latter case, the maximum degree of deformation may be comprised in the minimization criterion mentioned hereinbefore.

In effect, steps S1-S6 described hereinbefore can be regarded as shape recognition processing, which is applied to the volume data VD and based on the geometrical model GM of the fetal heart. This shape recognition processing extracts main anatomical parts of the fetal heart such as, for example, the heart chambers, the main vessels (thoracic aorta, thoracic arch, pulmonary arteries, vena cavae . . . ), the septa, the large valves. Stated otherwise, the volume data VD is segmented, whereby respective segments correspond with respective anatomic features. Flow information, which the ultrasound scanning assembly may provide by Doppler-mode processing, may assist this segmentation. This particularly applies to the chambers of the fetal heart and the main vessels. These anatomic features may be recognized more easily with the aid of flow information, which may be in the form of color.

In step S7 (MTCH=OK?), the feature extractor FEX determines whether a sufficiently good match between the effective geometrical model GM_{E} and the volume data VD has been obtained in step S6, or not. The feature extractor FEX may make this determination on the basis of a matching error that results from step S6. In case the smallest matching error that can be found between the volume data VD and the geometrical model GM exceeds a given threshold, the feature extractor FEX determines that no sufficiently good match has been obtained. The smallest matching error may depend on the maximum degree of deformation that is allowed to align the geometrical model GM with the volume data VD. In case a sufficiently good match has been obtained, the feature extractor FEX subsequently carries out step S9, which will be described hereinafter. In the opposite case, the feature extractor FEX subsequently carries out step S8.

In step S8 (MFI), the feature extractor FEX provides a match-failure indication signaling that no sufficiently good match has been found between the geometrical model GM and the volume data VD. The feature extractor FEX may apply this indication to the controller CTRL. In response, the controller CTRL may cause the display device DPL to display a warning message informing the operator that the matching process has failed. The warning message may further indicate the possible causes of this failure. The volume data VD may be of insufficient quality. Another cause is that the fetal heart suffers from a major defect. Once the warning message has been displayed, the controller CTRL may allow manual manipulations. Accordingly, the operator may inspect the volume data VD in a manual fashion. For example, the operator may define geometrical locations where slices SX are to be generated from the volume data VD. The warning message may assist the operator by indicating a location where matching was particularly difficult in the sense that matching errors were particularly important at this location. This information may itself be of diagnostic value to the operator.

In step S9 (ARD), which is carried out when a sufficiently good match has been obtained, the feature extractor FEX provides an anatomy-related description ARD of the volume data VD. As mentioned hereinbefore, the anatomy-related description ARD identifies respective segments in the volume data VD that correspond with respective anatomic features of the fetal heart, such as, for example, the heart chambers, the main vessels, the septa, and the large valves. A segment may be identified by means of a set of fields comprising a feature-identifying field and one or more geometrical-coordinate fields. In this example, the feature-identifying field specifies the anatomic feature concerned and the one or more geometrical-coordinate fields specify a region within the volume data VD that represents the anatomic feature concerned.

In step S10 (GEN_SX), the slice generator SLG generates a set of slices SX from the volume data VD based on the anatomy-related description ARD that the feature extractor FEX provides. To that end, the slice generator SLG may comprise a predefined algorithm that calculates respective slice locations from the anatomy-related description ARD so as to obtain a set of so-called standard views. A standard view of the fetal heart under examination may be compared with a standard view of another fetal heart, which may serve as a reference, or a reference model for the standard view. Standard views therefore allow relatively fast and reliable diagnosis. In conventional ultrasound systems, standard views can be obtained in a substantially manual fashion only, which involves a relatively great number of manipulations that an operator needs to carry out. This requires relatively high level of skill and experience and, nonetheless, this process is error-prone and relatively time-consuming. In an ultrasound system in accordance with the invention, such as the one described with reference to the figures, the feature extractor FEX allows an operator to find standard views more quickly and more reliably.

In step S11 (GEN_SGI), the controller CTRL, or the feature extractor FEX, may generate segment-related graphical information that can be overlaid on a slice. The segment-related graphical information may indicate respective anatomic features and their respective locations in the slice. For example, contours of the IV septa an AV valves, chambers, aortic arch, ductal arch, or the crux position of the heart may be indicated. The segment-related graphical information can be generated from the anatomy-related description ARD. The segment-related graphical information may comprise various colors, each of which indicates a particular anatomic feature.

Overlaid segment-related graphical information constitutes a tool that allows the operator to validate or correct slices SX that have been generated. The user may appreciate whether a slice is of sufficient quality, in particular in terms of positioning. In this respect it should be noted that the anatomy-related description ARD is subject to inaccuracies, which may be due to the following. There is a trade-off between, on the one hand, robustness of the matching process described hereinbefore in terms of, for example, immunity to noise, missing data, and anatomic variations, and, on the other hand, precision with which anatomic features are identified and their geometrical properties. An anatomic feature of interest can be subtle in terms of voxel variations and can be small relative to broker resolution. For example, outflow tract boundaries and small valves tend to show tenuously in the volume data VD.

In step S12 (DET_RAX), the controller CTRL, or the feature extractor FEX, may determine one or more axes of rotation for a particular slice on the basis of the anatomy-related description ARD. The one or more axes of rotation are typically related to anatomic features that have been identified in the matching process. For example, an axis of rotation may correspond with the long axis of the left ventricle, or the ascending aorta, or a straight part of any vessel. Such an axis of rotation, which is automatically determined, may assist the operator in adjusting the slice concerned. In this respect, it is noted that translating a slice, which may be regarded as “flying through” the volume data VD, is relatively intuitive. Rotation is more complex as it requires setting the axis of rotation and the angle of rotation around this axis. In order to manually achieve a given rotation, the operator has to compose a series of rotations around along various main axes, X, Y, and Z, and with respect to a center.

The controller CTRL, or the feature extractor FEX, may further use the anatomy-related description ARD for other operations relating to slice generation or adjustment. For example, a centerline of a vessel may be determined on the basis of the anatomy-related description ARD. A slice may be generated so that the slice is perpendicular to the centerline of the vessel concerned. What is more, a series of slices SX may be generated, which may be equidistantly spaced, along the centerline whereby each slice is perpendicular to the centerline of the vessel. Accordingly, a multi-slice view of a region of interest can be provided in automatic fashion.

In step S13 (DPL_SX&SGI&RAX), the display processor generates a display image DIS that comprises one or more slices SX. In the display image DIS, a slice may be overlaid with segment-related graphical information that has been generated as described hereinbefore with respect to step S11. The display image DIS may further visualize one or more axes of rotation and one or more centerlines that have automatically been determined as described hereinbefore with respect to steps S12 and S13. This may further assist the operator in evaluating and manipulating slices SX that are generated from the volume data VD.

In step S14 (ADJ?), the controller CTRL detects whether the operator has defined an adjustment for a particular slice, or not. The adjustment may be in the form of, for example, a rotation, which may involve one or more axes of rotation that have automatically been determined as described hereinbefore. The adjustments may also be in the form of, for example, a translation, which may be along a centerline that has automatically been determined as described hereinbefore. Notwithstanding the aforementioned, the adjustment may entirely be operator-defined in the sense that the anatomy-related description ARD does not play any particular role in defining the adjustment. In case the controller CTRL has not defined an adjustment for any slice, step S16 is subsequently carried out, which will be described hereinafter. In case the controller CTRL detects that the operator has defined an adjustment for a particular slice, the controller CTRL subsequently carries out step S15.

In step S15 (SXP), the controller CTRL generates a set of slice-extraction parameters on the basis of the adjustment that the operator has defined. The controller CTRL applies this set of slice-extraction parameters to the slice generator SLG. This can be regarded as a return to step S9. The slice generator SLG generates a slice from the volume data VD that is an adjusted version of the slice that was displayed in step S13. Segment-related information may be overlaid on this adjusted slice, which corresponds to step. The adjusted slice is included in a display image DIS, which corresponds to step S13.

In step S16 (SX=SVW?), the controller CTRL detects whether the operator has designated a slice as a standard view, which is to be stored for the purpose of, for example, reporting. In case the operator has not designated a slice, the controller CTRL may return to step S14. In case the controller CTRL detects that a standard view has been designated, the controller CTRL may subsequently carry out step S17.

In step S17 (ANN_SX), the controller CTRL automatically annotates the slice that has been designated as a standard view. The anatomy-related description ARD makes this possible. Annotation may provide several advantages, such as, for example, easing learning, helping non-expert users, and speeding up reporting. The controller CTRL preferably allows the operator to modify or complete annotations that have automatically been generated. The anatomy-related description ARD may further enable the controller CTRL to automatically select and activate appropriate measurement tools for measuring, for example, ventricular volumes and main vessel diameters. Measurements results obtained by such tools may complete the annotation. An annotated version of the slice, which constitutes a standard view, may be stored and automatically included in an appropriate report section.

### CONCLUDING REMARKS

The detailed description hereinbefore with reference to the drawings is merely an illustration of the invention and the additional features, which are defined in the claims. The invention can be implemented in numerous different ways. In order to illustrate this, some alternatives are briefly indicated.

The invention may be applied to advantage in numerous types of products or methods related to volumetric ultrasound imaging. For example, the invention may be applied in a portable computer, which is configured for volumetric ultrasound imaging purposes. The portable computer may interface with, for example, a dedicated ultrasound imaging module that comprises, for example, one or more beamformers as well as other circuits for applying activation signals to a probe and for processing reception signals from the probe. Such a dedicated ultrasound imaging module will typically comprise analog to digital converters and digital to analog converters.

The invention may be used to advantage in numerous types of examinations that involve ultrasound scanning Although the detailed description specifically mentions fetal heart examinations as an example, this by no means excludes other types of examinations, such as, for example, examinations of the fetal head, the female pelvis, as well as other clinical targets.

There are numerous ways of implementing a method or system in accordance with the invention, which involves searching for a best match between volume data and a geometrical model of an anatomical entity. For example, a module, which may be software-based, may be provided that allows an operator to indicate one or more an anatomic features in the volume data, in particular in geometric terms. This specifically applies to anatomic features that are generally easy to recognize, such as, for example, the crux of the heart, Referring to FIG. 1, these indications may serve as “seed” points, as it were, which can assist the feature extractor FEX or the slice generator SLG, or both, to implement operations defined in the claims and described hereinbefore. Accordingly, greater robustness may be achieved, although providing seed points potentially increases user interaction,

Although a drawing shows different functional entities as different blocks, this by no means excludes implementations in which a single entity carries out several functions, or in which several entities carry out a single function. In this respect, the drawings are very diagrammatic. For example, referring to FIG. 1, the feature extractor FEX and the slice generator SLG may be implemented by means of a single processor, which also implements the controller CTRL.

There are numerous ways of implementing functional entities by means of hardware or software, or a combination of both. As mentioned hereinbefore with reference to FIG. 1, the ultrasound scanning assembly USC, the feature extractor FEX and the slice generator SLG are functional entities that may each be implemented by means of a set of instructions that has been loaded into a programmable processor. In this respect, FIG. 1 can be regarded to represent a method, whereby the ultrasound scanning assembly USC represents an ultrasound scanning step, the feature extractor FEX represents a feature extraction step, and the slice generator SLG represents a slice generation step. Although software-based implementations of these functional entities have been mentioned, hardware-based implementations are by no means excluded. Hardware-based implementations typically involve dedicated circuits, each of which has a particular topology that defines operations, which the dedicated circuit concerned carries out. Hybrid implementations are also possible in the sense that a system, or a functional entity comprises therein, comprises one or more dedicated circuits as well as one or more suitably programmed processors.

There are numerous ways of storing and distributing a set of instructions, that is, software, which allows an ultrasound imaging system to operate in accordance with the invention. For example, software may be stored in a suitable medium, such as an optical disk or a memory circuit. A medium in which software stored may be supplied as an individual product or together with another product, which may execute software. Such a medium may also be part of a product that enables software to be executed. Software may also be distributed via communication networks, which may be wired, wireless, or hybrid. For example, software may be distributed via the Internet. Software may be made available for download by means of a server. Downloading may be subject to a payment.

The remarks made herein before demonstrate that the detailed description with reference to the drawings, illustrate rather than limit the invention. There are numerous alternatives, which fall within the scope of the appended claims. Any reference sign in a claim should not be construed as limiting the claim. The word “comprising” does not exclude the presence of other elements or steps than those listed in a claim. The word “a” or “an” preceding an element or step does not exclude the presence of a plurality of such elements or steps. The mere fact that respective dependent claims define respective additional features, does not exclude a combination of additional features, which corresponds to a combination of dependent claims.

1. An ultrasound imaging system comprising:
an ultrasound scanning assembly (USC) arranged to provide volume data resulting from a three-dimensional scan of a body;
a feature extractor arranged to find a best match between the volume data and a geometrical model of an anatomical entity, the geometrical model comprising respective segments representing respective anatomic features, so as to provide an anatomy-related description of the volume data, which identifies respective geometrical locations of respective anatomic features in the volume data; and
a slice generator for generating slices from the volume data, the slice generator being arranged to define respective slice locations from the anatomy-related description of the volume data so as to obtain a set of standard views of the anatomical entity of interest.

2. (canceled)

3. An ultrasound imaging system according to claim 1, the feature extractor being arranged to provide a match-failure indication in case a smallest matching error that can be found between the volume data and the geometrical model exceeds a given threshold.

4. An ultrasound imaging system according to claim 1, comprising:
a user interface arranged to allow an operator to define a minimization criterion that is to be used in finding the best match between the volume data and the geo metrical model.

5. An ultrasound imaging system according to claim 1, the feature extractor being arranged to obtain a reference slice that serves as a starting point in finding the best match between the volume data and the geometrical model.

6. An ultrasound imaging system according to claim 1, comprising:
a processor arranged to generate segment-related graphical information from the anatomy-related description, and to overlay the segment-related graphical information on a slice that has been generated from the volume data.

7. An ultrasound imaging system according to claim 1, comprising:
a processor arranged to determine at least one axis of rotation for a slice on the basis of the anatomy-related description, which axis of rotation can be displayed in association with the slice.

8. An ultrasound imaging system according to claim 1, comprising:
a processor arranged to generate annotations for a slice on the basis of the anatomy-related description, which annotations can be stored in association with the slice.

9. An ultrasound imaging system according to claim 1, the feature extractor being arranged to apply a deformation to the geometrical model in finding the best match between the volume data and the geometrical model.

10. An ultrasound imaging system according to claim 1, the feature extractor being arranged to obtain a maximum degree of deformation that can be applied to the geometrical model in finding the best match between the volume data and the geometrical model.

11. An ultrasound imaging system according to claim 1, the ultrasound scanning assembly being arranged to provide an indication of a time position within a biological cycle to which the volume data pertains, the feature extractor being arranged to apply the geometrical model as a function of the indication of the time position.

12. A method of ultrasound imaging involving an ultrasound scanning assembly arranged to provide volume data resulting from a three-dimensional scan of a body, the method comprising:
a feature ex traction step in which a best match between the volume data and a geometrical model of an anatomical entity is searched for, the geometrical model comprising respective segments representing respective anatomic features, so as to provide an anatomy-related description of the volume data, which identifies respective geometrical locations of respective anatomic features in the volume data; and
a slice generation step in which slices are generated from the volume data by defining respective slice locations from the anatomy-related description of the volume data so as to obtain a set of standard views of the anatomical entity of interest.

13. (canceled)

14. A computer program product for an ultrasound imaging system comprising
an ultrasound scanning assembly arranged to provide volume data resulting from a three-dimensional scan of a body:
a programmable processor,
the computer program product comprising a set of instructions, which when loaded into the programmable processor, enables the programmable processor to carry out a feature extraction step in which a best match between the volume data and a geometrical model of an anatomical entity is searched for, the geometrical model comprising respective segments representing respective anatomic features, so as to provide an anatomy-related description of the volume data, which identifies respective geometrical locations of respective an atomic features in the volume data; and
a slice generation step in which slices are generated from the volume data by defining respective slice locations from the anatomy-related description of the volume data so as to obtain a set of standard views of the anatomical entity of interest.
